(19) United States
(12) Patent Application Publication
(10) Pub. No.: US 2026/0090819 A1
(43) Pub. Date: Apr. 2, 2026

(54) Devices and Methods for Endoscopically Reconstructing Gastric Tissue

(71) Applicant: Michael Murray, Fallon, NV (US)

(72) Inventor: Michael Murray, Fallon, NV (US)

(21) Appl. No.: 19/346,716

(22) Filed: Oct. 1, 2025

Related U.S. Application Data
(60) Provisional application No. 63/702,191, filed on Oct. 2, 2024.

(51) Int. Cl. A61B 17/32; A61B 1/00; A61B 1/273; A61B 17/00; A61B 17/068

(52) U.S. Cl. CPC A61B 17/320016; A61B 1/00066; A61B 1/2733; A61B 17/0682; A61B 2017/00827

(57) ABSTRACT

A method of endoscopically reconstructing a stomach includes passing an end effector of an intraluminal treatment device through an esophagus and into a stomach; directing the end effector to a position adjacent a fold extending from a lesser curvature of the stomach; grasping the fold between a first jaw and a second jaw of the end effector; deploying fixation elements from the end effector and into the fold; and cutting through the fold adjacent to the fixation elements so as to form a passageway through the fold.

### CROSS-REFERENCE TO RELATED APPLICATIONS

This application claims the benefit of U.S. Provisional Application No. 63/702,191, filed on Oct. 2, 2024, the disclosure of which is hereby incorporated by reference in its entirety.

### BACKGROUND

The gastrointestinal (“GI”) tract is the system involved in the digestion and absorption of food and liquids. FIG. 1 depicts a portion of an exemplary GI tract, which includes the esophagus 10, stomach 20, and duodenum 30 (i.e., the first part of the small intestine). The gastroesophageal valve 12 (or lower esophageal sphincter) is located at the junction between the esophagus and the stomach, where it prevents the backflow of gastric acid into the esophagus and regulates the passage of food into the stomach. Further along the GI tract, the pylorus 23 of the stomach includes the pyloric valve 25 (or pyloric sphincter). The pyloric valve controls the release of partially digested food from the stomach into the duodenum, ensuring a regulated and gradual entry of food for efficient digestion and nutrient absorption.

The stomach is characterized by two primary curvatures: the greater curvature 22 and the lesser curvature 24, as shown in FIG. 1. The greater curvature is a relatively long convex arch extending from the fundus 21 of the stomach down to the pylorus. In contrast, the lesser curvature is relatively short, forms a concave arch extending from the fundus to the pylorus, and is generally located opposite of the greater curvature. During digestion, the greater curvature can expand significantly to accommodate food. Because of this, the portion of the stomach that includes the greater curvature is sometimes removed in bariatric surgeries (e.g., gastric sleeve surgery) to limit the stomach's capacity for expansion and reduce the amount of food it can hold.

While the GI tract has valves that regulate the passage of food into and out of the stomach, the stomach itself includes several musculo-mucosal folds that act as baffles to slow or inhibit the flow of food through the stomach. As shown in FIGS. 2 and 3, one such fold 26 (or baffle) may extend from the lesser curvature during digestion, potentially acting as a valve within the stomach. This fold 26 is typically located proximal to the pylorus at the incisura of the stomach and helps the stomach function as a large sedimentation chamber, allowing solids to layer out along the greater curvature, with fluids collected above the solid layers and gas collected above the fluids in the fundus.

As food is digested, the pH of the gastric acid within the stomach may increase. In response, the stomach may secrete additional gastric acid, which can collect as a layer of relatively low pH acid at the gastroesophageal junction. This acid layer 40 (or acid pocket) can cause acid reflux, and when produced chronically, it is characteristic of gastroesophageal reflux disease (“GERD”). Patients who have undergone bariatric surgery, particularly gastric sleeve procedures, have higher incidences of GERD than those who have not undergone such procedures. This is at least due to there being less space for gastric acid to escape into the duodenum between fold 26 and the reconstructed portion of the stomach where the greater curvature used to be.

### BRIEF SUMMARY OF THE DISCLOSURE

The present disclosure describes devices, systems, and methods for reconstructing the lesser curvature of the stomach to create a passage for excess stomach acid to escape into the lower intestine and reduce the incidence of acid reflux which can be particularly beneficial for patients with GERD.

In a first aspect of the present disclosure, a method of endoscopically reconstructing a stomach includes passing an end effector of an intraluminal treatment device through an esophagus and into a stomach; directing the end effector to a position adjacent a fold extending from a lesser curvature of the stomach; grasping the fold between a first jaw and a second jaw of the end effector; deploying fixation elements from the end effector and into the fold; and cutting through the fold adjacent to the fixation elements so as to form a passageway through the fold.

Additionally, the passageway may be formed such that it extends through the fold in a direction perpendicular to a length of the fold. Also, the cutting step may include moving a blade along the first jaw and through first and second sides of the fold. The cutting step may also include resecting a portion of the fold and removing the portion of the fold from the stomach. The elements may include staples, and the deploying step may include firing the staples through respective slots in the first jaw and into the fold.

Continuing with this aspect, the end effector may include a first configuration in which the intraluminal treatment device has a first maximum cross-sectional dimension, and a second configuration in which the intraluminal device has a second maximum cross-sectional dimension greater than the first maximum cross-sectional dimension. The passing step may be performed with the end effector in the first configuration. The method may also include transitioning the end effector from the first configuration to the second configuration while the end effector is positioned within the stomach. In the first configuration, the second jaw may be oriented relative to the first jaw up to about 180 degrees, and in the second configuration, the second jaw may be positioned opposite the first jaw and may be oriented less than 90 degrees relative to the first jaw. The end effector may be coupled to an elongate member. In the first configuration, the second jaw may be positioned within a recess of the elongate member, and the transitioning step may include rotating the second jaw about a hinge and out from the recess.

Further, the grasping step may include extending a grasping tool from between the first and second jaws, grasping the fold between opposed fingers of the grasping tool, and pulling the fold into a space between the first and second jaws. The end effector may be coupled to an elongate member, and the grasping tool may be extended through a lumen of the elongate member. The method may also include projecting an endoscope between the first and second jaws to visualize the esophagus and/or stomach from a perspective of the end effector. The end effector may be rotatably coupled to an elongate member via at least one hinge, and the directing step may include rotating the end effector about the at least one hinge relative to the elongate member. The intraluminal device may include a control handle coupled to a proximal end of the elongate member, and the control handle may be positioned external to the patient.

In another aspect of the present disclosure, an intraluminal treatment device may include an elongate member that has a proximal end and a distal end, and a control handle may be coupled to the proximal end of the elongate member. The control handle may have at least one control interface. The intraluminal treatment device may also include an end effector coupled to the distal end of the elongate member. The end effector may have a first jaw and a second jaw. The first jaw may have plurality of slots each of which may be configured for passage of a fixation element therethrough, a cutting guide, and a blade moveable along the cutting guide. The end effector may have a first configuration in which the intraluminal treatment device has a first maximum cross-sectional dimension configured for endoscopic passage through an esophagus into a stomach, and a second configuration in which the intraluminal device has a second maximum cross-sectional dimension greater than the first maximum cross-sectional dimension.

Additionally, the first jaw may have a free end and a fixed end, the second jaw may have a free end and a fixed end, and the free end of the second jaw may be further from the free end of the first jaw in the first configuration than in the second configuration. Also, the fixed end of the second jaw may be coupled to one of the first jaw and elongate member via a hinge. In the first configuration, the second jaw may be oriented up to about 180 degrees relative to the first jaw, and in the second configuration, the second jaw may be oriented less than 90 degrees relative to the first jaw. Furthermore, the distal end of the elongate member may include a recess extending axially therein. In the first configuration, the second jaw may be positioned within the recess, and in the second configuration, the second jaw may be positioned outside of the recess and in opposition to the first jaw. The plurality of slots of the first jaw may be staple slots configured for passage of a staple into tissue.

### BRIEF DESCRIPTION OF THE DRAWINGS

FIG. 1 is schematic cross-sectional view of a portion of a GI tract including a stomach in a first condition.

FIG. 2 is a schematic cross-sectional view of the GI tract of FIG. 1 with the stomach being in a second condition forming a fold.

FIG. 3 is a partial luminal view of the fold of FIG. 2.

FIG. 4A is a partial perspective view of an intraluminal treatment device according to an embodiment of the present disclosure and depicting an entry configuration.

FIG. 4B is a partial perspective view of the intraluminal treatment device of FIG. 4A depicting a deployment configuration.

FIG. 4C is a partial schematic view of a control handle of the intraluminal device of FIG. 4A.

FIG. 4D is a schematic elevational view of a first jaw of the intraluminal treatment device of FIG. 4A.

FIGS. 5-7 are schematic representations of exemplary cutting patterns of the intraluminal device of FIG. 4A.

FIGS. 8A-8D illustrate an endoscopic method of using the exemplary intraluminal device of FIG. 4A.

### DETAILED DESCRIPTION

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended.

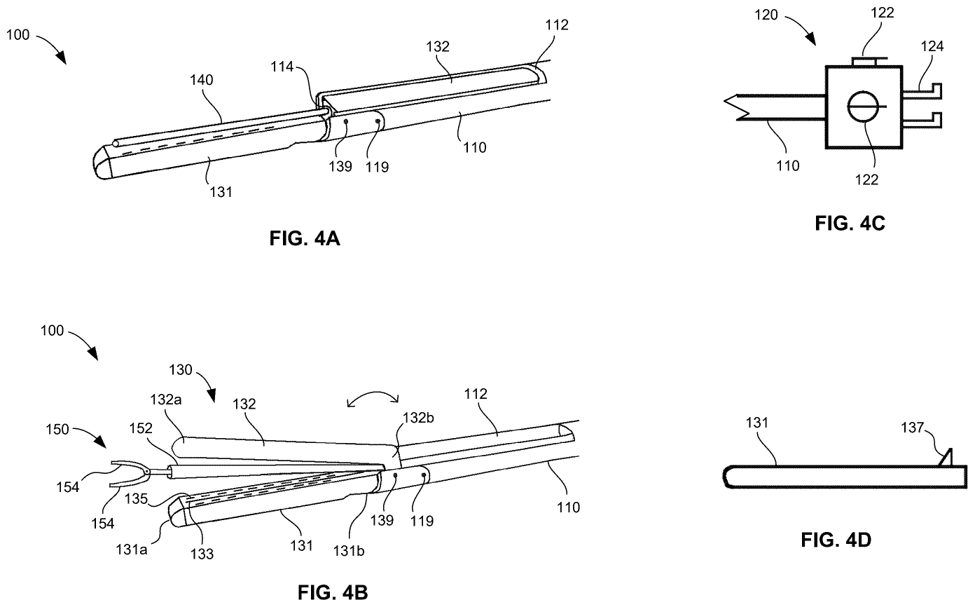

FIGS. 4A-4D depict an intraluminal treatment device 100 according to an embodiment of the present disclosure. Intraluminal device 100 generally includes an elongate member 110, a control handle 120 coupled to a proximal end of elongate member 110, and an end effector 130 coupled to a distal end of elongate member 110.

Elongate member 110 (or guide shaft) is configured to extend endoscopically through a patient's esophagus to the stomach. Elongate member 110 may be rigid along its entire length. Alternatively, elongate member 110 may be flexible along a portion of its length or along an entirety of its length. As shown in FIGS. 4A and 4B, elongate member 110 may include a recess 112 extending axially from the distal end of elongate member 110 toward the proximal end thereof. Such recess 112 may be configured to receive a portion of end effector 130 for reducing the profile of device 100. For example, recess 112 may be configured to receive a jaw of end effector 130 while device is in an entry configuration, as shown in FIG. 4A and as described in more detail below.

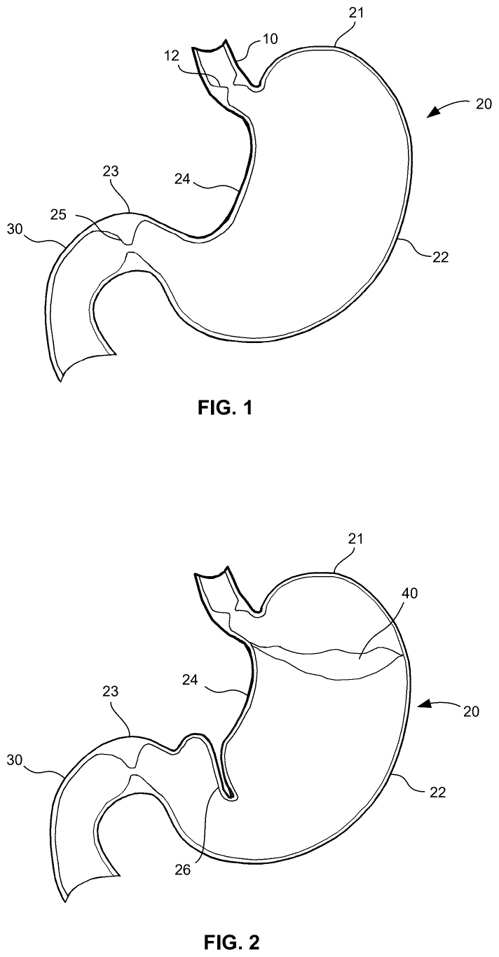

Control handle 120 may include one or more control interfaces 122, 124. Such control interfaces 122, 124 may include knobs 122 and/or levers 124, for example. In some embodiments, knobs 122 may be configured to bend and/or steer one or more portions of elongate member 110 within a patient's GI tract, such as the GI tract shown in FIGS. 1 and 2. For example, knobs 122 may be coupled to pull-wires (not shown) which, when tensioned, may produce a bend in elongate member 110 at one or more locations along its length. Additionally, or alternatively, one or more levers 124 may control one or more elements of end effector 130 to perform desired operations on targeted tissue, as described in more detail below.

End effector 130 may be pivotably connected to the distal end of elongate member 110 via hinge 119. Hinge 119 may have multiple degrees of freedom (e.g., via a universal joint or living hinge) or a single degree of freedom. Although a single hinge 119 is depicted, end effector 130 may be coupled to elongate member 110 via a plurality of hinges 119 each having one or more degrees of freedom.

End effector 130 may also be provided with one or more functionalities. In this regard, end effector 130 may be configured as a stapler and, as such, may include a first jaw 131 and a second jaw 132. First jaw 131 (or first member) may have a free end 131a and a fixed end 132b. First jaw 131 may house a plurality of staples (or fixation elements), such as in a modular cartridge, for example, and may define a plurality of staple slots 135 through which staples are ejected into targeted tissue. Such slots 135 may axially extend between free end 131a and fixed end 131b and may be arranged in one or more offset rows of staple slots 135. In the embodiment depicted, first jaw 131 includes two rows of staple slots 135. However, in other embodiments, first jaw 131 may include one row of staple slots 135 or three to six rows of staple slots 135, for example. The rows of staple slots 135 may be arranged parallel to each other, and the slots 135 of each row may be longitudinally aligned or longitudinally offset from each other. Such longitudinally offset relationship may help close off any openings between adjacent staples within the same row.

Second jaw 132 (or second member) is disposed opposite first jaw 131 and, as such, may operate as a backstop or anvil to the staples being fired from first jaw 131. Second jaw 132 may have a free end 132a and a fixed end 132b. Fixed end 132b may be pivotably connected to elongate member or first jaw via hinge 139. Hinge 139 may have a single degree of freedom, and second jaw 132 may be pivotable about hinge 139 up to about 180 degrees, as illustrated in FIGS. 4A and 4B. In this regard, end effector 130 may have a first configuration (or entry configuration) (see FIG. 4A) in which second jaw 132 is positioned within recess 112 and is oriented about 180 degrees relative to first jaw 131. Additionally, when in the first configuration free end 132a of second jaw 132 may point in a proximal direction, while a free end of second jaw 132 may point in a distal direction. In the entry condition, intraluminal treatment device 100 defines a first maximum cross-sectional dimension configured for passage endoscopically through the GI tract, and more particularly through the esophagus to the stomach. End effector 130 may also have a second configuration (or deployment configuration) (see FIG. 4B) in which second jaw 132 is oriented less than 90 degrees relative to first jaw 131 and, as such, is positioned outside of recess 112 and for pinching or grasping tissue between first and second jaws 131, 132 enabling deployment of staples into the targeted tissue. Additionally, when in the second configuration, free end 132a of second jaw 132 may point in a distal direction along with free end 131a of first jaw 131. In other words, free end 132a of second jaw 132 is positioned further from free end 131a of first jaw in the first configuration than in the second configuration. As described in more detail below, end effector 130 may be transitioned to the second configuration within the stomach of a patient and may define a second maximum cross-sectional dimension of intraluminal device which is greater than the first maximum cross-sectional dimension.

While end effector 130 depicts first and second jaws 131, 132, it is contemplated that end effector 130 may also include a third and fourth jaw, for example, which may function similarly to first and second jaws 131, 132. Also, while second jaw 132 is depicted as being pivotable up to about 180 degrees, it is contemplated that in some embodiments, first jaw 131 may instead be pivotable up to about 180 degrees and positionable within a corresponding recess similar to recess 112 in elongate member 110.

End effector 130 may also be configured as a cutting device or resection device. In this regard, end effector 130 may include one or more guide slots 133 (or cutting guide) (see FIG. 4B) and a blade 137 (or cutting member) (see FIG. 4D) moveable within or along guide slot 133. Such blade 137 may be configured to move concurrently with the firing of staples from first jaw 131 or subsequently to the firing of staples. In the embodiment depicted, cutting slot 133 may extend in a longitudinal direction between rows of staple slots 135. Thus, staples deployed from staple slots 135 may be used to secure resected tissue at opposite sides of guide slot 133.

It is also contemplated that the one or more control interfaces 122, 124 on control handle 120 may be configured to operate end effector 130. For example., knobs 122 or one or more levers 124 may be configured coapt first and second jaws 131, 132 and compress tissue between jaws 131, 132 prior to stapling. Additionally, or alternatively, control interfaces 122, 124 may initiate stapling and/or cutting.

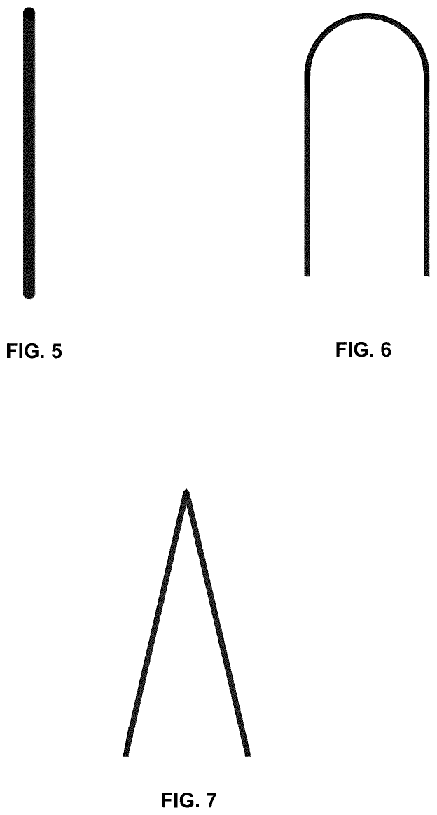

FIG. 5 illustrates the cutting and stappling pattern formed by end effector 130 of FIGS. 4A and 4B which, as shown, is a linear pattern. In another example, as shown in FIG. 6, end effector 130 may be configured with a U-shaped resection track or slot along which a blade cuts a U-shaped pattern in tissue grasped between first and second jaws 131, 132. A corresponding U-shaped piece of tissue may be resected away from the tissue and removed from the GI tract via end effector 130. Staples may be placed in one or more rows along the outside of the U-shaped pattern and may optionally be placed along the inside of the U-shaped pattern. FIG. 7 depicts another exemplary pattern which is a V-shaped pattern. Resection and stapling of tissue along this pattern may be performed similarly to that of the U-shaped pattern of FIG. 6. The patterns depicted herein are merely exemplary of the many possible patterns that can be achieved. However, it should be understood that in the patterns of FIGS. 6 and 7 a correspondingly shaped piece of tissue may be removed from the GI tract, while in the pattern of FIG. 5, a linear cut may be made without the removal of any tissue.

Intraluminal device 100 may include additional tools which may be moveable into and removable from a space between first and second jaws 131, 132. In this regard, elongate member 110 may include at least one lumen 114 configured to receive such tools, and jaws 131, 132 may define a space therebetween sufficient to receive any one of such tools. For example, as shown in FIG. 4A, an endoscope 140 (or gastroscope) may extend along at least a portion of first jaw 131 at least while end effector 130 is in the first configuration. In other words, endoscope 140 may pass between first and second jaw 131, 132. Such passage may occur when first and second jaws 131, 132 are in the first and second configurations. This allows the operator to view the intraluminal space from the perspective of end effector 130. Additionally, endoscope 140 may be used to steer device 100 to the target location. The space between first and second jaw 131, 132 in the first configuration may be more limited than in the second configuration. As such, second jaw 131 may have notches, grooves, or through-openings that provide clearance for the passage of endoscope 140 when in the first configuration.

Another exemplary tool that may be implemented in intraluminal device is a grasping tool 150. Grasping tool 150 may extend between first and second jaws 131, 132 at least while end effector 130 is in the second configuration and may be axially extendable and retractable. A separate cannula 152 (see FIG. 4B) may be provided to help facilitate the grasping tool's delivery to the space between first and second jaws 131, 132 and movement therebetween. Such cannula 152 may also be used by endoscope 140 for passage between first and second jaws 131, 132. Grasping tool 150 may have opposable fingers 154 that may be moveable to grasp targeted tissue so that, when grasping tool 150 is retracted, grasping tool 150 may position the targeted tissue between first and second jaws 131, 132. Grasping tool 150 and/or endoscope 140 may have controls located on control handle 120 or they may have controls separate from control handle 120. In embodiments in which their controls are separate, control handle 120 may include one or more lumens for reception of endoscope 140 and/or grasping tool 150 into elongate member 110.

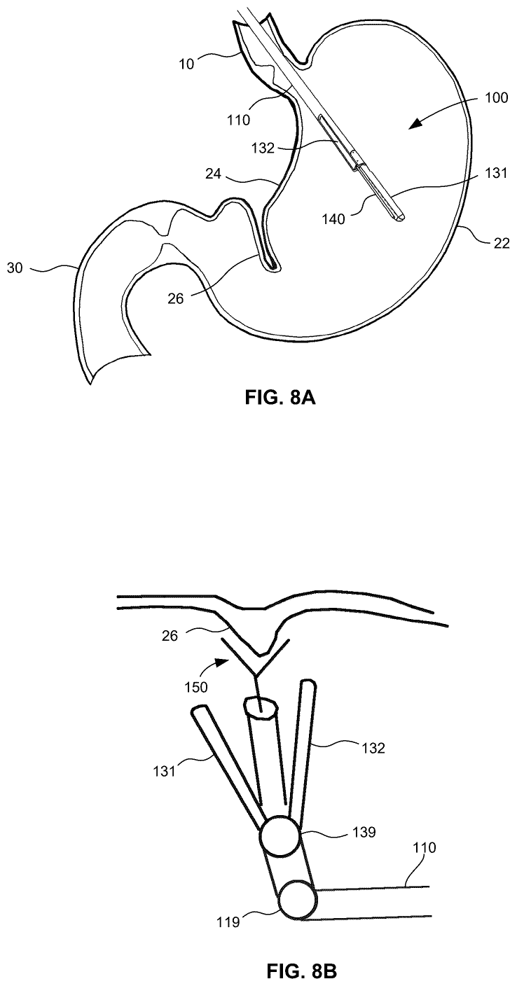

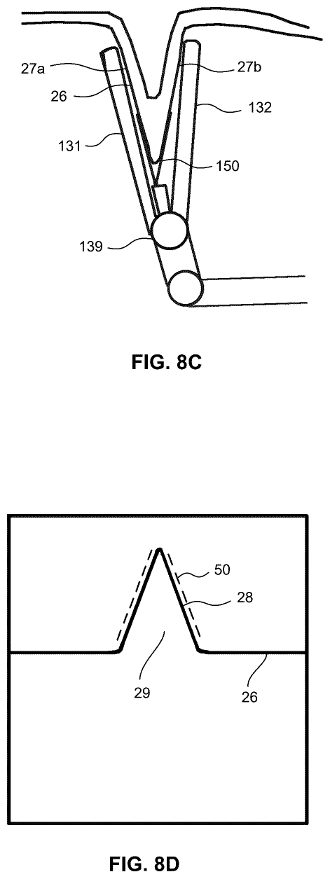

In addition to that described above, various other operations are now described and with reference to FIGS. 8A-8D. It should be understood that the following operations do not have to be performed in the exact order as described. Instead, various steps may be handled in a different order or simultaneously. Steps may also be omitted or added unless otherwise stated.

As shown in FIG. 8A, intraluminal device 100 may be placed in the entry configuration which is the lowest profile configuration of device 100. In this regard, second jaw 132 is rotated up to about 180 degrees relative to first jaw 131 such that second jaw 132 is positioned within recess 112 of elongate member 110. Additionally, endoscope 140 may be extended through elongate shaft 110 and out of the distal end thereof between first and second jaws 131, 132. Intraluminal device 100 may then be inserted through a patient's esophagus 10 and into the stomach 20.

Once end effector 130 is positioned within stomach 20, end effector 130 may be transitioned to the deployment configuration. In this regard, second jaw 132 may be rotated about hinge 139 so that second jaw 132 is positioned in opposition to first jaw 131 and is less than 90 degrees relative thereto. This may be achieved by operating one of the controls on control handle 120, such as moving one of levers 124 in a proximal-distal direction.

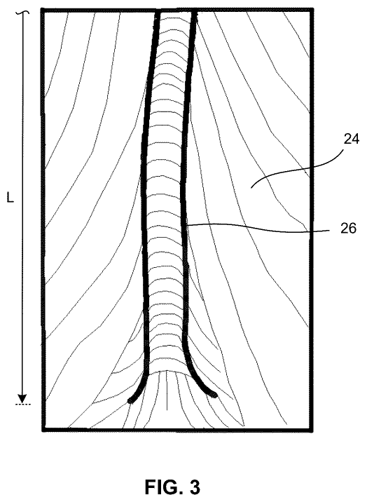

Once end effector 130 is positioned in the second configuration, end effector 130 may be steered or otherwise directed toward fold 26 extending from the lesser curvature 24 of stomach 20 proximal to pylorus 23 and at the incisura of stomach 20. In other words, fold 26 of stomach 20 may extend from lesser curvature 24 and be located between pylorus 23 and esophagus 12, but generally closer to pylorus 23 than esophagus 12, as illustrated in FIG. 2. Such fold 26 may define a length L extending in a sagittal plane, as shown in FIG. 3. End effector 130 is directed toward fold 26 which may be achieved by at least rotating end effector 130 about hinge 119 relative to elongate member 110 such that end effector 130 is angled relative to elongate member 110, as shown in FIG. 8B.

When end effector 130 is in position adjacent to fold 26, fold 26 may be grasped directly by first and second jaws 131, 132. Alternatively, grasping tool 150 may be extended out from between jaws 131, 132 toward fold 26, and fingers 154 may be actuated to grasp fold 26. Once grasped, grasping tool 150 may be retracted so that fold 26 is stretched out between first and second jaws 131, 132, as illustrated in FIG. 8C. In this regard, fold 26 may form a first side 27a and a second side 27b. First jaw 131 may be positioned at first side 27a of fold 26, and second jaw 132 may be positioned at second side 27b of fold 26, as shown in FIG. 8C. Endoscope 140 may be used to verify that the tissue 26 is sufficiently positioned between first and second jaws 131, 132.

Once the desired amount of tissue is positioned between first and second jaws 131, 132, jaws may be closed onto the respective sides 27a-b of fold 26 and the staples 50 may be fired into the tissue 26 through first and second sides 27a-b. Simultaneously or subsequently to firing staples 50, blade 137 may be actuated so that it travels down guide slot 133 and cuts through first and second sides 27a-b of fold 26. As illustrated in FIG. 8D, which is a sagittal view of fold 26, blade 137 may cut between the rows of staples 50 so as to form a passageway 29 (or notch) in fold 26 through which gastric acid can escape during digestion into pylorus 23 and into duodenum 30. Passageway 29 may be formed perpendicular to or otherwise transverse to the length L of fold. As shown, the cut may be of the linear pattern of FIG. 5 or of the V-shaped pattern of FIG. 7. Although the cut may be linear, the passageway 29 may form a V-shape when stomach 20 is stretched. Where a V-shaped passageway 29 is formed, the corresponding V-shaped resected tissue may continue to be grasped by jaws 131, 132 and/or by grasping device 150 and withdrawn from the GI tract along with intraluminal device 100. As shown in FIG. 8D, fold 26 remains folded as secured by staples 50. Staples 50 prevent gastric acid from escaping into the abdominal cavity.

Although intraluminal device 100 is described above being configured to deploy staples 50, other fixation elements may be utilized to secure opposing sides 27a-b of fold 26. For example, one or more sutures may be utilized in lieu of staples 50. In such embodiment, end effector 130 may include one or more suture needles which may be configured to penetrate tissue and apply a suture to the tissue in a similar pattern.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. A method of endoscopically reconstructing a stomach, the method comprising:
passing an end effector of an intraluminal treatment device through an esophagus and into a stomach;
directing the end effector to a position adjacent a fold extending from a lesser curvature of the stomach;
grasping the fold between a first jaw and a second jaw of the end effector;
deploying fixation elements from the end effector and into the fold; and
cutting through the fold adjacent to the fixation elements so as to form a passageway through the fold.

2. The method of claim 1, wherein the passageway extends through the fold in a direction perpendicular to a length of the fold.

3. The method of claim 1, wherein the cutting step includes moving a blade along the first jaw and through first and second sides of the fold.

4. The method of claim 1, wherein the cutting step includes resecting a portion of the fold and removing the portion of the fold from the stomach.

5. The method of claim 1, the fixation elements include staples, and the deploying step includes firing the staples through respective slots in the first jaw and into the fold.

6. The method of claim 1, wherein:
the end effector includes a first configuration in which the intraluminal treatment device has a first maximum cross-sectional dimension, and a second configuration in which the intraluminal device has a second maximum cross-sectional dimension greater than the first maximum cross-sectional dimension, and
the passing step is performed with the end effector in the first configuration.

7. The method of claim 6, further comprising transitioning the end effector from the first configuration to the second configuration while the end effector is positioned within the stomach.

8. The method of claim 7, wherein, in the first configuration, the second jaw is oriented relative to the first jaw up to about 180 degrees, and in the second configuration, the second jaw is positioned opposite the first jaw and is oriented less than 90 degrees relative to the first jaw.

9. The method of claim 8, wherein:
the end effector is coupled to an elongate member,
in the first configuration, the second jaw is positioned within a recess of the elongate member, and
the transitioning step includes rotating the second jaw about a hinge and out from the recess.

10. The method of claim 1, wherein the grasping step includes extending a grasping tool from between the first and second jaws, grasping the fold between opposed fingers of the grasping tool, and pulling the fold into a space between the first and second jaws.

11. The method of claim 10, wherein the end effector is coupled to an elongate member, and the grasping tool is extended through a lumen of the grasping tool.

12. The method of claim 1, further comprising projecting an endoscope between the first and second jaws to visualize the esophagus and/or stomach from a perspective of the end effector.

13. The method of claim 1, wherein the end effector is rotatably coupled to an elongate member via at least one hinge, and the directing step includes rotating the end effector about the at least one hinge relative to the elongate member.

14. The method of claim 13, wherein the intraluminal device includes a control handle coupled to a proximal end of the elongate member, the control handle being positioned external to the patient.

15. An intraluminal treatment device, comprising:
an elongate member having a proximal end and a distal end;
a control handle coupled to the proximal end of the elongate member and having at least one control interface; and
an end effector coupled to the distal end of the elongate member and having a first jaw and a second jaw, the first jaw having plurality of slots each being configured for passage of a fixation element therethrough, a cutting guide, and a blade moveable along the cutting guide,
wherein the end effector has a first configuration in which the intraluminal treatment device has a first maximum cross-sectional dimension configured for endoscopic passage through an esophagus into a stomach, and a second configuration in which the intraluminal device has a second maximum cross-sectional dimension greater than the first maximum cross-sectional dimension.

16. The device of claim 15, wherein:
the first jaw has a free end and a fixed end,
the second jaw has a free end and a fixed end, and
the free end of the second jaw is further from the free end of the first jaw in the first configuration than in the second configuration.

17. The device of claim 16, wherein the fixed end of the second jaw is coupled to one of the first jaw and elongate member via a hinge.

18. The device of claim 15, wherein, in the first configuration, the second jaw is oriented up to about 180 degrees relative to the first jaw, and in the second configuration, the second jaw is oriented less than 90 degrees relative to the first jaw.

19. The device of claim 15, wherein:
the distal end of the elongate member includes a recess extending axially therein,
in the first configuration, the second jaw is positioned within the recess, and
in the second configuration, the second jaw is positioned outside of the recess and in opposition to the first jaw.

20. The device of claim 19, wherein the plurality of slots of the first jaw are staple slots configured for passage of a staple into tissue.
